(19) United States
(12) Patent Application Publication
(10) Pub. No.: US 2014/0323785 A1
(43) Pub. Date: Oct. 30, 2014

(54) CATALYST COMPOSITE FOR DEHYDROGENATION OF HYDROCARBONS AND METHOD OF PREPARATION THEREOF

(71) Applicant: Reliance Industries Limited, Mumbai, Maharashtra (IN)

(72) Inventors: Sharad Vasuderao Lande, Akola (IN); Venkata Satya Bhaskara Sita Rama Murthy Katravulapalli Veera, Hyderabad (IN); Sreedharan Unnikrishnan, Vadodara (IN); Nagesh Sharma, Vadodara (IN); Shashank Vaidya, Thane (IN); Rajeshwer Dongara, Vadodara (IN); Krishnamurthy Ramaswamy Konda, Vadodara (IN)

(73) Assignee: RELIANCE INDUSTRIES LIMITED, Mumbai, Maharashtra (IN)

(21) Appl. No.: 14/359,226

(22) PCT Filed: Nov. 20, 2012
(86) PCT No.: PCT/IN2012/000757

§ 371 Date: May 19, 2014

(30) Foreign Application Priority Data
Nov. 21, 2011 (IN) 3190/MUM/2011

(51) Int. Cl. B01J 31/26; C07C 5/32

(52) U.S. Cl. CPC B01J 31/26; C07C 5/325; C07C 2523/42; C07C 2523/46; C07C 2523/14
USPC 585/660; 502/222; 502/223; 502/168

(57) ABSTRACT

The present invention provides a dehydrogenation catalyst composite that is capable of providing a dehydrogenated hydrocarbon product characterized by a bromine number of at least 19. for hydrocarbons. The dehydrogenation catalyst of the present invention comprises a nano-sized complex containing a Group VIII component; a group IVA component and a sulfur containing capping agent; an alkali component; a halogen component; and a support with an inner core of alpha alumina and an outer layer comprising a mixture of gamma alumina and delta alumina.

### FIELD OF INVENTION

The present invention relates to catalysts for use in the process for producing linear alkylbenzene. More particularly, the present invention relates to a novel catalyst composition and a method for the manufacture thereof. The present invention also relates to a process for the manufacture of unsaturated hydrocarbons i.e., mono-olefins from saturated hydrocarbons i.e., paraffins through the process of dehydrogenation.

### BACKGROUND OF INVENTION AND PRIOR ART

Dehydrogenation of saturated hydrocarbons or paraffins, specifically C2-C20 paraffins, is an important petrochemical process through which a number of useful unsaturated hydrocarbons are manufactured. These unsaturated hydrocarbons are the olefinic monomers, such as ethylene, propylene, butenes, butadiene, styrene and straight chain mono olefins of carbon number range C6-C20, which find extensive applications in the production of a variety of plastics, synthetic rubber and detergents etc. Furthermore, dehydrogenation of naphthenes and paraffins are the most important reactions during catalytic reforming processes, practiced worldwide, for the production of aromatics (BTX) and high octane gasoline.

### PRIOR ART

Catalyst composites comprising a group VIII metal component along with an additional metal component such as tin, germanium, rhenium or mixtures of such additional metal components have been disclosed in U.S. Pat. No. 3,632,503, U.S. Pat. No. 3,755,481, U.S. Pat. No. 3,878,131, U.S. Pat. No. 3,682,838, U.S. Pat. No. 3,761,531, U.S. Pat. No. 3,558,477, U.S. Pat. No. 3,562,147, U.S. Pat. No. 3,584,060, U.S. Pat. No. 3,897,368.

A metal component containing catalysts wherein platinum is impregnated onto a calcined support, are disclosed in U.S. Pat. Nos. 3,755,481; 3,761,531; and 4,104,317.

The catalysts disclosed in another U.S. Pat. No. 4,077,912 consists of an essentially inert support having a strongly adherent outer coating of active catalytic material. FIG. 2 of this patent clearly advocates the advantages of coated supports for exothermic reactions.

U.S. Pat. No. 4,430,517 discloses the use of a catalyst composite for C2-C30 hydrocarbons for dehydrogenation reaction consisting Pt, Sn and alkali component potassium wherein the atomic ratio of Pt/K is more than 10 and is about 15-25 wt %. The catalyst needs sulfiding and is tested for isobutane dehydrogenation.

U.S. Pat. No. 4,495,308 discloses two-region spherical catalysts. The catalyst described in this patent is a spherical catalyst for hydroprocessing hydrocarbonaceous feedstocks. The catalyst consists of an inner region having less than 5% of its port volume contributed by macropores and having between 5 and 30 weight percent total catalytic metals, and an outer region characterized by having more than 10% of its pore volume contributed by macropores and having between 1 and 15 weight percent catalytic metals.

U.S. Pat. No. 4,465,789 teaches hydrotreating catalyst support having dual pore structure. Hydrotreating catalysts are made on supports having a core of alumina having predominantly micropore structure, surrounded by a rind of different alumina having at least 25% of total pore volume in macropores.

U.S. Pat. No. 4,595,673 teaches the uses of Pt, Sn and either of In, Ga, or Tl, halogen compounds and mixtures of alkali compounds i.e., Lithium and potassium in the range of 0.05-10.0 wt %.

Indian Patent No. 180035 teaches the use of a dehydrogenation catalyst with alkali metal Na as a promoter along with Pt and Sn supported on dual pore alumina support.

Indian Patent No. 166585 discloses the conversion of hydrocarbons, especially, the dehydrogenation of hydrocarbons containing C2-C30 carbon atoms using a catalyst comprising platinum, tin and bifurcated alkali metal components consisting lithium and potassium on porous gamma alumina, with a low level of halogen component i.e., less than 0.1 wt %. The alkali metal component varies from 0.05-3 wt % potassium, based on the composite. This patent discloses the use of mixtures of alkali metals comprised Lithium and potassium. However, it is silent on the use of the specific combination of alkalis i.e. sodium and lithium.

A catalytic composite comprising a Group VIII noble metal co-formed with a Group IVA metal component, and which also comprise an alkali metal or alkaline earth metal component, and an alumina support is disclosed in U.S. Pat. No. 4,672,146

Catalyst based on supported platinum, wherein the distribution of the platinum is limited to the external surface of the support is disclosed in U.S. Pat. No. 4,716,143. However, one of the shortcomings of the distribution of the platinum on the external surface is that it can only rarely produce homogeneous platinum/modifier atomic ratios on the particle scale (nanometer). Additionally, the chances of diffusional limitations in the catalyst grain (extragranular diffusion) on account of the over-concentration of active phase are very high. Such diffusional limitations reduce the overall reaction yield.

U.S. Pat. No. 4,762,960 discloses a catalytic composite comprising a platinum group metal component; a modifier metal component selected from the group consisting of a tin component, germanium component, rhenium component and mixtures thereof; an alkali or alkaline earth metal component or mixtures thereof, an optional halogen component, and an optional catalytic modifier component on a refractory oxide support having a nominal diameter of at least about 850 microns.

U.S. Pat. No. 4,827,072, and U.S. Pat. No. 4,716,143 describe a dehydrogenation catalyst composition and hydrocarbon dehydrogenation process. The catalytic composite of these patents comprise a platinum group metal component; a modifier metal component selected from the group consisting of a tin component, germanium component, rhenium component and mixtures thereof; an optional alkali or alkaline earth metal component or mixtures thereof, an optional halogen component, and an optional catalytic modifier component on a refractory oxide support having a nominal diameter of at least about 850 microns. The distribution of the platinum group metal component is such that the platinum group component is surface-impregnated where substantially the entire platinum group metal component is located at most within a 400 micron exterior layer of the support. The effective amount of the modifier metal component is uniformly dispersed throughout the refractory oxide support.

U.S. Pat. No. 5,012,027 discloses the dual profile surface impregnated dehydrogenation catalyst and process. The catalyst composite consists a Pt group component Ir, Os, or mixture, a second component Sn, supported on alumina. The catalytic component is characterized in that both the components are surface impregnated. Though the patent discusses the use of various mixtures of alkali metals, it does not teach the use of any particular alkali metal mixtures and its method of impregnation to achieve specific advantages.

U.S. Pat. No. 5,358,920 discloses the use of catalyst for C6-C16 paraffin dehydrogenation which consists Pt as the active component, tin as the second component, large pore diameter-alumina with dual pore diameter distribution as the support and sodium as the promoter. The patent does not describe the use of combination of alkali metals as promoter.

U.S. Pat. No. 5,516,740 teaches catalyst comprising thin shell of catalytically active material bonded onto an inert core. A catalyst is disclosed comprising a thin outer shell of catalytic material bonded to an inner core of catalytically inert material. The catalyst is made by coating a catalytically inert core such as alpha alumina, with a thin layer of finely divided catalytically active material in slurry of colloidal boehmite/pseudo boehmite then calcining to convert the boehmite/pseudo boehmite into gamma alumina thereby bonding it to the inert core.

Indian Patent No. 178059 and U.S. Pat. No. 5,677,260, teach a process for the preparation of a catalyst composite incorporating a predetermined gradient of active elements within its spatial geometry for use in the dehydrogenation of paraffins to the corresponding monoolefins. Catalysts compositions taught in these patents consist of noble metal Pt, tin, Indium, iron and lithium on porous gamma alumina.

U.S. Pat. No. 5,200,382 teaches comprising a thin outer shell of catalytic material bonded to an inner core of catalytically inert material. The catalyst is made by coating a catalytically inert core such as alpha alumina, with a thin layer of finely divided catalytically active material in slurry of colloidal boehmite/pseudo boehmite then calcining to convert the boehmite/pseudo boehmite into gamma alumina thereby bonding it to the inert core.

Indian Patent No. 163412 discloses the conversion of hydrocarbons, especially dehydrogenation of paraffins having from 2 to 5 or more carbon atoms, to the corresponding mono-olefins or diolefins with catalyst comprising platinum, tin and alkali metal components, potassium, caesium and their combinations, with porous alumina as the carrier material.

Indian Patent No. 179677 and U.S. Pat. No. 6,700,028 disclose the conversion of hydrocarbons, especially dehydrogenation of paraffins having from 2 to 30 carbon atoms, to the corresponding mono-olefins or di-olefins by employing a catalyst comprising platinum, tin and alkali metal components, potassium with porous alumina as the carrier material.

U.S. Pat. No. 6,417,135 teaches regeneratable supported catalysts with porous support useful in the dehydrogenation of hydrocarbons. Catalysts made according to this patent possess a unique pore size distribution which provides a favorable balance of selectivity, activity, and thermal stability.

U.S. Pat. No. 6,498,280 describes a catalyst comprising at least one support and it contains 0.31% by weight of platinum, 0.32% by weight of tin, 0.13% by weight of indium, 0.35% by weight of lithium and 0.09% by weight of chlorine. The patent does not describe the use of combination of alkali metals as promoter.

U.S. Pat. No. 6,177,381, U.S. Pat. No. 6,280,608 and U.S. Pat. No. 6,756,515 disclose a layered catalyst composition, a process for preparing the composition and processes for using the composition. The catalyst composition comprises an inner core such as alpha-alumina, and an outer layer bonded to the inner core composed of an outer refractory inorganic oxide such as gamma-alumina. The outer layer has uniformly dispersed thereon a platinum group metal such as platinum and a promoter metal such as tin. The composition also contains a modifier metal such as lithium.

### OBJECTS OF THE INVENTION

It is an object of the present invention to provide a catalyst with low diffusion resistant support.

It is another object of the present invention to provide a dehydrogenation catalyst composite having an improved activity and selectivity.

It is still another object of the present invention to provide a dehydrogenation catalyst composite having dimensional stability i.e. it is not prone to hydrothermal degradation of the catalyst support and catalyst thereon during prolonged operations.

It is a further object of the present invention to provide a catalyst with inherent acidity that ensures minimal dehydration of isopropyl alcohol.

It is a still further object of the present invention to provide a catalyst with a combination of promoters and moderators which shows high conversion of paraffins to the corresponding mono olefins with high selectivity to mono-olefins.

It is yet another object of the present invention to provide a catalyst that involves minimal coke formation.

It is still another object of the present invention to provide a catalyst composite wherein the active metal component is uniformly distributed on the surface of the catalyst support.

### SUMMARY OF THE INVENTION

In a first aspect of the present invention, there is provided a dehydrogenation catalyst composite for hydrocarbons comprising:

a nano-sized complex containing a Group VIII component; a group IVA component and a sulfur containing capping agent;

an alkali component;

a halogen component; and

a support with an inner core of alpha alumina and an outer layer comprising a mixture of gamma alumina and delta alumina and;

The catalyst composite of the present invention is capable of providing a dehydrogenated hydrocarbon product characterized by a bromine number of at least 19.

Typically, the Group VIII component is selected from the group consisting of platinum and iridium and a combination thereof.

Typically, the particle size of the nano-sized complex is in the range of 0.4 to 2 nm.

Preferably, the particle size of the nano-sized complex is in the range of 0.4 to 1 nm.

Typically, the nano-sized complex is characterized by UV spectra as shown in FIG. 6.

Typically, the sulphur containing capping agent is selected from the group consisting of thioglycolic acid and thiomalic acid (TMA). Preferably, the sulphur capping agent is thiomalic acid (TMA).

Typically, the Group IV A component is tin.

Typically, the proportion of delta alumina in the out core of the support is in the range of 15-25% with respect to the weight of the total alumina present in the core.

The catalyst composite of the present invention exhibits an X-ray powder diffraction pattern as shown in FIG. 2.

Typically, the surface area of the support ranges between 20 to 200 m2/g.

Typically, the halogen component is selected from the group consisting of fluorine, chlorine, bromine and iodine.

Typically, the proportions of Group VIII component, group IVA component, alkali component, group VIA component, and the halogen component in the catalyst composite are in the ranges between 0.01 to 5.0 wt %, 0.01 to 5 wt %, 0.01 to 15 wt %, 0.01 to 1 wt % and 0.05 to 0.07 wt % respectively.

Typically, the Group VIII component is distributed on the outer layer of the support.

Typically, the Group IV A component is distributed uniformly on the outer layer of the support.

Typically, the amount of tin expressed as Sn:Pt atomic ratio is in the range from 0.1-10 and preferably, from 0.6-4.0 in the final form of the catalyst.

In accordance with a second aspect of the present invention, there is provided a process for the manufacture of a catalyst composite of the present invention comprising the following steps:

preparing a spheroidal alumina support; subjecting the spheroidal alumina support to an impregnation with a first alkali metal using a first alkali metal precursor; drying and calcining the first alkali metal impregnated support; re-impregnating the first alkali impregnated support with a group VIII component; a group IVA component, a group VIA component; a halogen component, a second alkali metal and a capping agent using their respective precursors; drying and calcining the re-impregnated support and contacting the re-impregnated support with a gas stream until the halogen content of the catalyst decreases to a predetermined level to obtain a partially de-halogenated composite; reducing the partially de-halogenated composite by treating it with a stream of gas under chemically reducing conditions to obtain a catalyst composite.

Typically, the method step of preparing a spheroidal alumina support comprises selecting an inert alpha alumina core sphere of average 1.2 mm diameter; coating the alpha alumina core with activated alumina powder and a binder till it attains an average diameter size of 1.8 mm; hydrating the coated core, heating the hydrated core in the presence of air to obtain spheroidal alumina support containing a mixture of gamma, delta and theta alumina in the outer layer.

Typically, the hydrated core is heated at a temperature in the range of about 845° C. to 855° C.

Typically, the first alkali metal impregnated support is calcined at a temperature in the range of 630° C. to 650° C. for four hours.

Typically, the re-impregnated support is treated with a gas stream until the halogen content of the catalyst is reduced to 0.07wt %.

Typically, the group VIII component is a mixture of platinum and iridium.

Typically, the Group VIII component is platinum and it is impregnated by using chloroplatinic acid salt as its precursor by at least one technique selected from the group consisting of ‘co-gelling with the support’ and ‘chemical vapour deposition’.

Typically, the first and second alkali metals are lithium and sodium respectively and the precursors for the first and the second alkali metals are lithium nitrate and sodium chloride respectively.

Typically, the halogen is chlorine and it's precursor during impregnation is HCl solution.

Typically, the halogen component is impregnated together with or before the addition of the group VIII component.

Typically, the Group IVA component is tin and it is impregnated on the support using aqueous solution of its precursor, stannous chloride.

Typically, the molar ratio of the first alkali metal and the second alkali metal is about 0.3:1.

Typically, the first alkali metal precursor is lithium nitrate coverts the gamma alumina in the outer layer of the support to lithium aluminate.

Typically, the first and second alkali components are present as oxides, or in combined form with the support material or with the other catalytic components.

Typically, the first and second alkali metal components are lithium and sodium respectively and their proportions in the final catalytic composite ranges between 0.05 to 2 wt %, and 0.05 to 1.0 wt % respectively.

Typically, said support is substantially refractory in the hydrocarbon reaction medium.

Typically, said treatment with a stream of gas under chemically reducing conditions is performed either in-situ or off-site.

Typically, said treatment with a stream of gas under chemically reducing conditions is performed off-site followed by purging with high purity inert gas stream at elevated temperature in the range of 400-600° C., at high GHSV of 100-10000 h⁻¹ followed by cooling in the same inert gas stream for passivation.

Typically, said treatment is carried out at GHSV in the range of 2000-5000 h⁻¹,

Typically, the temperature of the catalyst bed is raised slowly at 30°-40° C./h, preferably, 30° C./h from ambient to between 400-500° C., preferably, 450-480° C. for a duration of 0.5-10 h, preferably, 2-3 h and a pressure sufficient to maintain the throughput of the gas stream through the catalyst bed is applied.

In accordance with a third aspect of the present invention there is provided a process for the dehydrogenation of hydrocarbons which comprises contacting said hydrocarbons with a catalytic composite of the present invention under conditions suitable for the dehydrogenation reaction. Preferably, the hydrocarbon feed is contacted with said catalyst at a temperature in the range of 400 to about 800 C, pressure of from about 0.1 to 10 atmospheres and a LHSV of about 0.1 to 100^{-h}.

### BRIEF DESCRIPTION OF THE ACCOMPANYING DRAWINGS

FIG. 1 provides an XRD Pattern of the reference alumina used for preparing the catalyst.

FIG. 2 provides a XRD pattern of the RPDC-10 catalyst of the present invention as prepared in Example 1 and 2.

FIG. 3 shows the HR-TEM nano-particle size measurement of the platinum particle present in the fresh catalyst of the present invention.

FIG. 4 shows the BR-TEM nano-particle size measurement of the spent catalyst in accordance with the present invention.

FIG. 5 shows the characteristics UV spectrum of the Pt—Sn-TMA complex present in the catalyst of the present invention.

FIG. 6 shows the impact of addition of TMA on the UV absorbance during the formation of the Pt—Sn-TMA complex.

### DETAILED DESCRIPTION

In accordance with a first aspect of the present invention there is provided a catalyst composite particularly useful for dehydrogenating hydrocarbons, especially paraffins from 2 to 15 or more carbon atoms to the corresponding mono olefins, diolefins and to aromatics, especially to higher percentage of mono-olefins.

The catalyst composite of the present invention is capable of providing a dehydrogenated hydrocarbon product characterized by a bromine number of at least 19. Bromine number (or “bromine index”) is the amount of bromine in milligrams absorbed by 100 grams (3.5 oz) of a sample. The number indicates the degree of unsaturation in the sample. Bromine number value is an important indicator of the percentage conversion of n-paraffin.

The catalyst composite of the present invention comprises:

a nano-sized complex containing a Group VIII component; a group IVA component and a sulphur containing capping agent;

an alkali component;

a halogen component; and

a support with an inner core of alpha alumina and an outer layer comprising a mixture of gamma alumina and delta alumina.

In the context of the present invention, the term “a Group VIII metal component” means platinum or platinum and iridium.

Typically, the atomic ratio of alkali metal component to the Group VIII component is more than 50.

Regarding the support material, the preferred alumina support material is spherical with a particle size ranging between 1.2-2.00 mm in diameter. It is preferably a porous absorptive support with high surface area of from about 20 to 200 m²/g. The support material is refractory in the hydrocarbon reaction medium. The support present in the catalyst composite of the present invention comprises an inner core of inert alpha alumina. The outer layer of the support comprises a combination of gamma, delta and theta alumina. The proportion of delta alumina in the outer layer of the support in the catalyst ranges between 15% to 25%. The high proportion of delta alumina enhances the selectivity of the catalyst composite towards olefins and lowers aromatics formation.

The particles size of the nanosized particles of this complex ranges between 0.4 to 2 nm and preferably between 0.4 to 1 nm. The group VIII component present in this complex is a combination of platinum and iridium. The amount of Pt present is enough to be catalytically effective and ranges from 0.01-5 wt %, preferably, between 0.1-0.5 wt % in the final form of the catalyst; while the amount iridium in the final catalyst composite ranges between 0.05-0.5 wt %.

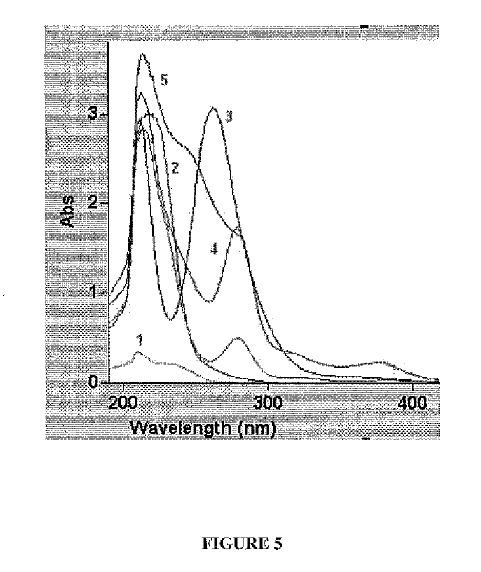

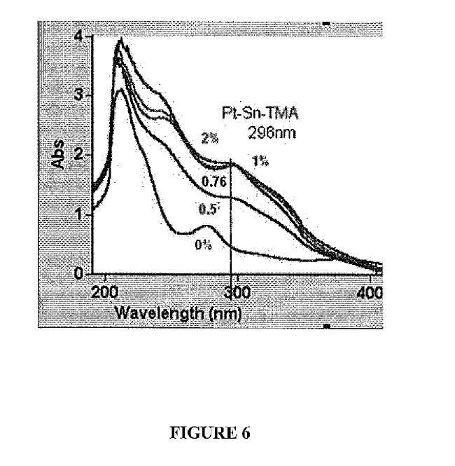

The nano-sized complex containing a Group VIII component; a group IVA component and a sulphur containing capping agent present in the catalyst composite of the present invention has been characterized by its specific UV absorption spectrum as shown in FIGS. 5 and 6. In FIG. 5, the individual spectrums of reagents (TMA, SnCl₂, CPA, Pt—Sn complex) used for the formation of the Pt—Sn-TMA complex are shown by curves 1 to 4. The curve indicated by number 5 shows the characteristic UV spectra for the Pt—Sn-TMA complex of the present invention. FIG. 6 shows the effect of addition of TMA solution (0, 0.5, 0.76, 1.0 and 2%) and the characteristic UV absorption of the Pt—Sn-TMA complex of the present invention.

The nano-sized complex containing a Group VIII component; a group IVA component and a sulphur containing capping agent formed on the outer layer of the support significantly contributes to the enhancement in selectivity as well as activity of the catalyst composite of the present invention. TMA present in the nano-sized complex generates nano-particles of platinum metal by complexation. Furthermore, it also masks the platinum metal in the last method step of preparation of the catalyst composite i.e. during reduction. This kind of masking ensures high activity of the platinum during the hydrogenating process.

It has been observed that the presence of iridium in combination with platinum results into a catalyst composite that shows higher bromine number. Thus, the presence of iridium is associated with the improvement in selectivity and activity of the catalyst composite of the present invention.

The Pt component in the active state of the catalyst is present substantially in the elemental state with or without chemical interaction with some of the other metallic components viz. tin, sodium and the support, depending upon the method of preparation. This Pt component is desirably distributed in the gamma alumina coating. Concentration gradient of platinum is from surface richness to decreasing while going to centre of the sphere. Platinum in the core portion of the sphere is negligible.

The group IV A component, of the said catalyst is preferably tin, which is present partly in elemental state with or without chemical interaction with Pt or wholly in oxidic state, with or without interaction with the support, when the latter is gamma alumina. It is preferably distributed uniformly throughout the geometry of the outer layer only.

The tin content in the catalyst, as Sn:Pt atomic ratio may range from 0.1-10 and preferably, from 0.6-4.0 in the final form of the catalyst.

Preferably, the alkali component is well dispersed through out the catalytic composite. The alkali or alkaline earth component generally comprise about 0.05 to about 2 wt % of lithium and from about 0.05 to 1.0 Wt % of sodium, calculated from elemental basis of the final catalytic composite. Preferably the catalyst comprises from about 0.05 to about 2.0 wt % lithium and from about 0.05 to about 4.0 wt % sodium.

The catalytic composite of the present invention also contains a halogen component. The halogen component may be fluorine, chlorine, bromine or iodine or mixture thereof. Chlorine and bromine are the preferred halogen components. The halogen component is generally present, in a combined state with the porous support material and the alkali component. Preferably, the halogen component is well dispersed throughout the catalyst composite. The halogen component may comprise from more than 0.07 wt % calculated on an elemental basis of the final catalytic composite.

In a second aspect of the present invention, there is provided a process for preparation of the catalyst composite of the present invention. The process for preparation of the catalyst composite of the present invention comprises the following steps:

preparing a spheroidal alumina support; subjecting the spheroidal alumina support to a first impregnation with a first alkali metal using a first alkali metal precursor; drying and calcining the first alkali metal impregnated support; re-impregnating the first alkali impregnated support with a group VIII component; a group IVA component, a group VIA component; a halogen component, a second alkali metal and a capping agent using their respective precursors; drying and calcining the re-impregnated support and subjecting the re-impregnated support to controlled de-halogenation to obtain a catalyst composite.

The method step of preparing a spheroidal alumina support comprises selecting an inert alpha alumina core sphere of average 1.2 mm diameter; coating the alpha alumina core with activated alumina powder and a binder till it attains an average diameter size of 1.8 mm; hydrating the coated core, heating the hydrated core in the presence of air to obtain spheroidal alumina support containing a mixture of delta and theta alumina. The coated and hydrated core is heated at a temperature in the range of about 845° C. to 855° C. The proportion of the delta and theta alumina in the support of the present invention ranges between 10% to 25% with respect to the weight of the total alumina. Activated alumina once heated at 850° C., gives phase mixture of delta and theta alumina (Reference: Industrial Alumina Chemicals, C. Mishra, ACS Monograph, 1986).

The decreased gamma alumina concentration per given volume results in decreased acidity entailing lowering of isomerisation and oligomerization reactions.

In accordance with one embodiment of the invention, inert alpha alumina core sphere of avg. 1.2 mm diameter is used as core. The support is prepared by growing the core sphere by coating it with activated alumina powder and binder in rotating pan till it attains avg 1.8 mm diameter size. The material is then hydrated to attain strength and dried. The alumina carrier is then subjected to high temperature treatments during its preparation to convert into non-acidic support coat of lithium aluminates. Typically, the coated core is then heated at 850° C. temperature in the presence of air.

After the preparation of the support, it is subjected to sequential impregnation. The impregnation process parameters are carefully selected to achieve uniformity and also desired distribution. Preparation of complex of Pt and Sn and TMA (thiomalic acid) is critical. Preferably, the group IVA component is Sn and the halogen component is Cl.

The Pt component may be impregnated before, together with or after the other metallic components, as thermally decomposable salts, chloroplatinic acid being preferable or is cogelled with the support at the support preparation stage or incorporated from the vapour phase by chemical vapour deposition. Halogen, preferably, Cl as HCl solution may be added together with or before the addition of the Pt component.

This Group IV A component may be co gelled with the support at the initial stage of support preparation or impregnated on to the formed support before or after the stage of drying or calcination of the latter, before, together with or after impregnation of the other metallic components, preferably on the formed, finished support, together with the other metallic components. This component may be incorporated into the composite as thermally decomposable salt solutions, preferably aqueous solutions of their chlorides. Optionally halogen, preferably Cl, may be added during impregnation of this component, preferably when all components are impregnated together in a single step.

Precursors for Platinum and group IV A component Sn are preferably hexa chloro platinum (IV) and stannous chloride which are complexed with capping agents like mercaptanes, thiols, and surfactants. The above solution is treated at elevated temperature to maximise the possibilities for complexation. To disperse the platinum and tin into nano particles surfactants like ethylene glycol, linear alkyl benzene sulphates are added.

Among the capping agents, thiomalic acid and among the surfactants ethylene glycol is preferred. The synergy results into narrow distribution of nano particles. Heating at 60 to 70 C for 1 h results into complex, which upon adding ethylene glycol results into colloidal nano solution of platinum and tin. The capping agents chosen are, such that they leave group VIA component sulphur. This results into innovative recipe with better selectivity towards the desired mono-olefins production.

Regarding the mixture of alkali components, better results have been obtained with a combination of lithium with sodium wherein the molar ratio of sodium to lithium is about 0.3:1, although good results are also obtained at atomic ratios above this value. Lithium may be incorporated as lithium nitrate and treated in oxidising environment to convert gamma alumina into lithium aluminate. Sodium precursor used is sodium chloride, which is impregnated separately along with platinum and tin complex. Alkali components exist in the final form, in an oxidation state above that of metal. The alkali component may be present as the oxide, or in combined form with the carrier material or with the other catalytic components.

The alkali component may be incorporated in the catalytic composite in any suitable manner such as, for example, by co-precipitation or co-gelation, by ion exchange or impregnation or by like procedures either before, while or after other components are incorporated. A preferred method of incorporating the alkali component being to impregnate the carrier material with a solution of first lithium nitrate and then sodium chloride along with Pt and Sn.

The halogen composite may be incorporated in any suitable manner, either during the preparation of the carrier material or before while or after other catalytic components are incorporated. For example, the alumina sol utilized to form the preferred aluminium carrier material may contain halogen and thus contribute at least some portion of the halogen content in the final catalyst composite. Also the halogen or a portion thereof may be added to the catalyst composite during the incorporation of the carrier material with other catalyst components, for example by using chloroplatinic acid to impregnate the platinum component. Also the halogen component or a portion thereof may be added to the catalyst component by contacting the catalyst with the halogen or compound, solution, suspension or dispersion containing the halogen before or after other catalyst components are incorporated in the support material. Suitable components containing the halogen include acids containing the halogen, for example, hydrochloric acid.

The above prepared composite is further contacted with a gas stream preferably air, containing between 0.01-10.0 mol % H₂O and preferably, about 5-8 mol %, until the halogen content of the catalyst decreases to the desired level ranging from 0.01-3.0 wt % and preferably 0.05-0.3 wt %. Complete leaching of the halogen component results into low acidity, but severely affects the sintering rate of platinum. This is particularly avoided in the process of the present invention wherein pre-determined halogen content is maintained in the final catalyst composite, which ensures good platinum sintering stability with minimal or no isomerisation or cracking reactions.

As a last step of the method of catalyst preparation as embodied in this disclosure, the catalyst resulting from the forgoing steps is contacted with a stream of gas under chemically reducing conditions. This step may be performed either in-situ or off-site, the latter requiring an additional step, of purging with high purity inert gas stream at elevated temperature 400-600° C., at high GHSV 2000-5000 h-1 followed by cooling in the same inert gas stream for passivation. An essential prerequisite of the reduction of the catalyst is drying and purging O₂ from the surface of the catalyst by its treatment, at elevated temperature with an inert gas stream free from hydrocarbon, S or S containing compounds, moisture and O₂ prior to treatment of the catalyst with reducing gas. In the reduction step, pure H₂ free from hydrocarbons, S or S containing compounds and moisture, all of which should be less than 5 vol ppm each is used. Further, the said treatment is carried out at high GHSV ranging from 100-10000 h-1, preferably 2000-5000 h⁻¹. The temperature of the catalyst bed is raised slowly at 30°-40° C./h, preferably 30° C./h from ambient to between 400-500° C., preferably 450-480° C. for a duration of 0.5-10 h, preferably 2-3 h and a pressure sufficient to maintain the throughput of the gas stream through the catalyst bed is applied.

In a third aspect of the present invention, there is also provided a process for dehydrogenating the hydrocarbons by using the catalyst composite of the present invention. In accordance with the process of dehydrogenating of the present invention, the paraffinic hydrocarbons are contacted with the catalytic composite of the present invention in a dehydrogenation zone maintained at specified conditions. The feed composition is contacted with a catalyst bed either in a fixed bed or moving bed; however a fixed bed system is preferred. In this system the feed stream is preheated to the required temperatures before it is being fed to the catalyst bed. The feed stream may be passed either upward, downward or in radial fashion.

The paraffinic feed includes hydrocarbons from 2 to 20 or more carbon atoms. The catalyst composite of the present invention is particularly useful for paraffins having 2 to 15 or more carbon atoms to the corresponding mono olefins. The reaction conditions include a temperature range of 400 to about 800 C, pressure of from about 0.1 to 10 atmospheres and a LHSV of about 0.1 to 100 h⁻¹.

The dehydrogenatable paraffins is mixed with a diluent material, preferably hydrogen before being passed to the dehydrogenation zone. Hydrogen to hydrocarbon mole ratio is maintained in the range of 0.1:1 to about 40:1 however the preferred range is 1:1 to 10:1.

Water or material which decomposes during reaction temperature to give water is added to the reaction medium either continuously or intermittently in an amount to provide 100-20000 wt ppm of the hydrocarbon feed stream. Preferred water content based on the combined feed is 300 ppm V.

The following examples are introduced to further describe the catalyst and the process of the invention.

### EXAMPLE 1

Preparation of Catalyst Support

Inert alpha alumina core sphere of avg. 1.2 mm diameter were used as core. The particle was grown further by coating activated alumina powder and binder in rotating pan till it attained avg. 1.8 nun diameter size. The material was then hydrated to attain strength and dried. It was then heated at 850° C. temperature in the presence of air. Activated alumina upon heating at 850° C., gave phase mixture of delta and theta alumina (Reference: Industrial Alumina Chemicals, C. Mishra, ACS Monograph, 1986).

### EXAMPLE-2

Preparation of Catalyst Composite of Catalyst

Employing a two-step impregnation of a spheroidal coated alumina support as prepared in example 1, a catalyst composite was prepared with the following composition by adopting incipient wetness technique:

TABLE 1
Catalyst

| Pt | Sn | Ir | Na | Li | Cl | TMA |
|---|---|---|---|---|---|---|
| (wt %) | (wt %) | (wt %) | (wt %) | (wt %) | (wt %) | (wt %) |
| 0.17 | 0.20 | 0.05 | 0.30 | 0.20 | 0.20 | 0.05 |

In the first step of impregnation, a solution of LiNO₃ to give the loading as mentioned in the above table was employed to impregnate the support by wet impregnation; thereafter the support thus impregnated was dried and calcined at 640 C/4 h. Second impregnation was carried out with the salt solutions of Pt, Sn, Ir and Na. The precursors used are H₂PtCl₆, SnCl₂, Ir Cl₃ NaCl, HCl and TMA. The re-impregnated support was once again dried and calcined and then subjected to a controlled de-halogenation step in order to reduce the chlorine content thereof to 0.20% by weight.

Test Data:

1. Effect of the Presence of Iridium on Bromine Number.

Bromine number for the catalyst as prepared in accordance with example 1 and 2 was found out. Another catalyst was prepared by following the process provided in examples 1 and 2 except that it did not comprise iridium. The catalyst comprising iridium and the other one devoid of iridium were analysed and their respective bromine numbers were found out. The comparative bromine numbers of these catalysts are provided in Table 2.

TABLE 2

| Catalyst | Bromine Number |
|---|---|
| RPDC-10 (Catalyst | 21 |
| composition without Iridium) | |
| RPDC-10 (Catalyst | 23 |
| composition with Iridium) | |

It was found out the catalyst of the present invention containing iridium (as prepared in Example 1 and 2) showed a better bromine number as compared to the catalyst without iridium.

Alumina Phases in RPDC-10 (Catalyst of the Present Invention)

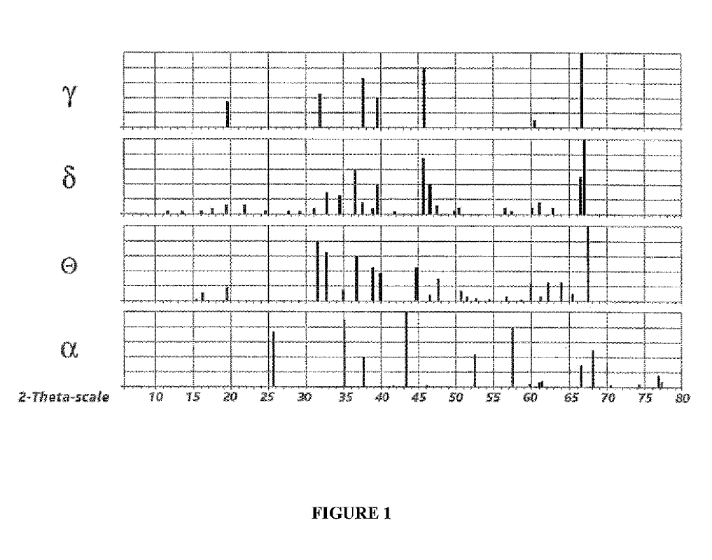

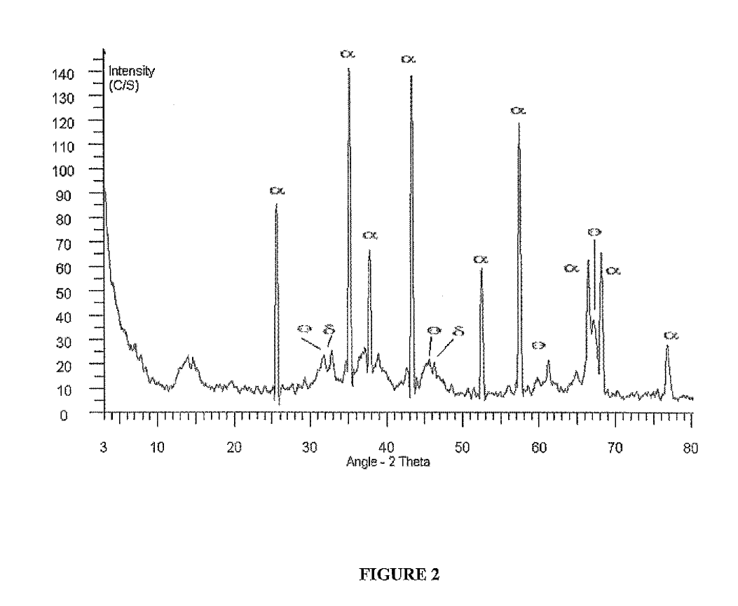

RPDC-10, the catalyst of the present invention as prepared in example 1 and 2 comprises a mixture of alpha alumina (contributed by core) and delta & theta alumina (contributed by outer layer). The reference profiles of alumina phases and XRD pattern of RPDC-10(Catalyst of the present invention) are shown in FIG. 1 and FIG. 2. Ref: Standard Transition Aluminas. Electron Microscopy Studies P. Souza Santosa, H. Souza Santos*b, S. P. Toledob Materials Research, Vol. 3, No. 4, 104-114, 2000.

2. Effect of TMA (Capping Agent) on RPDC-10 Catalyst Activity (Bromine Number Data)

The activity of RPDC-10 catalyst, the catalyst of the present invention as prepared in examples 1 and 2 by using TMA shows improved activity. The bromine number value for the catalyst of the present invention is 22 while it is 16 for the catalyst that is prepared without employing TMA. This is shown in Table 3 given below:

TABLE 3
Activity of RPDC-10 catalyst with TMA

| Catalyst | Bromine Number (Activity) |
|---|---|
| RPDC-10 (Catalyst | 16 |
| Preparation without TMA) | |
| RPDC-10 (Catalyst | 22 |
| Preparation with TMA) | |

3. Crystallite Size of the Platinum Metal in Case of the Catalyst of the Present Invention:

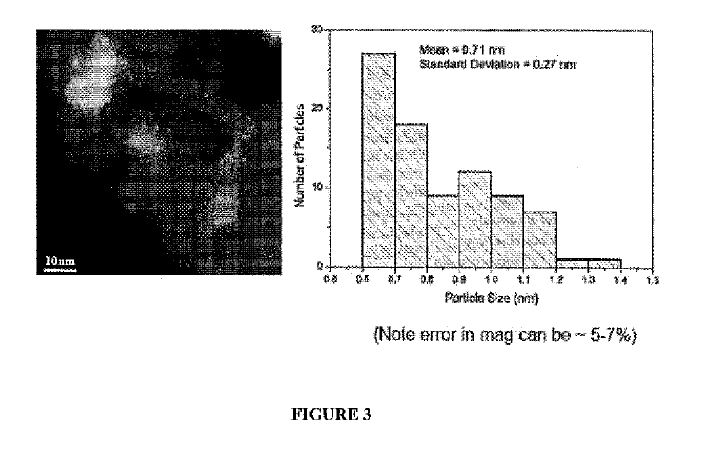

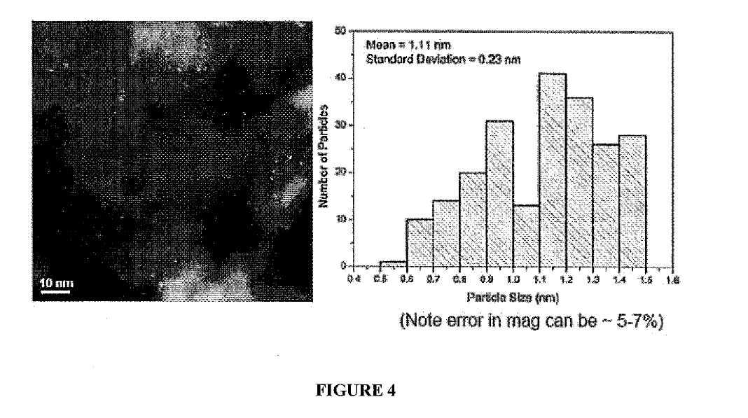

The particle size of the platinum particles in case of the catalyst in accordance with the present invention was measured after its preparation and after its use (spent catalyst). The average particle size for the catalyst of the present invention as found out by HR-TEM was 0.71 nm. This is shown in FIG. 3. In case of spent catalyst, the average platinum particle (FIG. 2) size was found to be 1.11 nm. This is shown in FIG. 4. The percentage of platinum particles in catalyst of the present invention (RPDC-10) after its preparation i.e fresh catalyst and its use i.e. spent catalyst by HR-TEM as shown in Table 4.

TABLE 4
Percentage of Pt particles in RPDC-10
fresh and spent catalyst by HR-TEM.

| Platinum | RPDC- 10 fresh | RPDC-10 spent |
|---|---|---|
| particle | catalyst Percentage | catalyst, Percentage |
| size, nm | of Pt particles, % | of Pt particles, % |
| 0.5-0.6 | 0 | 0.5 |
| 0.6-0.7 | 32.6 | 4.6 |
| 0.7-0.8 | 20.9 | 6.8 |
| 0.8-0.9 | 10.5 | 9.1 |
| 0.9-1.0 | 14. | 14.2 |
| 1.0-1.1 | 10.5 | 5.9 |
| 1.1-1.2 | 9.3 | 18.7 |
| 1.2-1.3 | 1.1 | 16 |
| 1.3-1.4 | 1.1 | 11.9 |
| 1.4-1.5 | 0 | 12.3 |

(Note error can be ~2-5%)

4. Performance Comparison of Layered RPDC-10 Catalyst of the Present Invention Verses Unisphere DHC-8 Catalyst (Prior Art Catalyst) at Commercial Scale

The activity of the catalyst of the present invention and prior art catalyst (DHC-8) was studied. The catalyst of the present invention and the prior art catalyst were evaluated for dehydrogenation of C10-C13 normal paraffins, carried out in a pilot plant reactor with product separation facility. The reactant was passed through catalyst bed containing 3 ml of catalyst. Two different sets of experiments were carried out by varying the pressure (one set at 1 atm. pressure and the other set at 20 psig.) Similarly the feed flow was varied so as to get the LHSV for first set at 30 h⁻¹ and for second set at 20 h⁻¹ respectively.

Hydrogen as diluent was added to the reaction to provide a molar ratio of Hydrogen to Hydrocarbon 6:1. The reaction temperature was 450° C. for the first set and 455-480° C. for 2nd set of experiments. The results of these tests for various catalysts are presented in FIGS. 1-5.

Activity is defined as a measure of the catalyst's ability to convert reactants into products at a specified set of reaction/process parameters viz. temperature, pressure, LHSV, reactant feed composition at given time on stream.

Selectivity is defined as the moles of desired product(s) formed per mole of reactant converted, at specified set of process parameters as described above.

Stability is defined as a measure of the rate of change in activity and selectivity with time on stream, a smaller rate implying higher stability. Most hydrocarbon conversion processes follow a “temperature policy” rather than a space time policy for maintaining reactant conversion at the desired level, wherein the reactor temperature is raised to compensate for the decrease in conversion due to catalyst deactivation. Hence reactor temperature is taken as indication of activity and stability.

A comparison between the activity of the catalyst in accordance with the present invention and the prior art catalyst (DHC-8) is provided herein below in Table 5.

TABLE 5
Performance comparison data

| | DHC-8 | RPDC-10 |
|---|---|---|
| Catalyst | (Prior Art) | (Present Invention) |
| Days on Stream | 37 | 65 |
| Catalyst Yield, MT of LAB/Kg | 5.83 | 6 |
| of catalyst | | |
| Specific consumption of NP | 0.811 | 0.775 |
| MT/MT of LAB | | |
| Specific generation of Light | 0.090 | 0.053 |
| End + H₂ MT/MT of LAB | | |
| Average LAB Production, | 172.93 | 174.98 |
| MT/Day | | |

While certain embodiments of the inventions have been described, these embodiments have been presented by way of example only, and are not intended to limit the scope of the inventions. Variations or modifications to the design and construction of this invention, within the scope of the invention, may occur to those skilled in the art upon reviewing the disclosure herein. Such variations or modifications are well within the spirit of this invention. The accompanying claims and their equivalents are intended to cover such forms or modifications as would fall within the scope and spirit of the invention.

The numerical values given for various physical parameters, dimensions and quantities are only approximate values and it is envisaged that the values higher than the numerical value assigned to the physical parameters, dimensions and quantities fall within the scope of the invention and the claims unless there is a statement in the specification to the contrary.

1-38. (canceled)

39. A dehydrogenation catalyst composite for hydrocarbons comprising:
a nano-sized complex containing a Group VIII component, a group IVA component and a sulfur containing capping agent;
an alkali component;
a halogen component selected from the group consisting of fluorine, chlorine, bromine and iodine; and
a support with an inner core of alpha alumina and an outer layer comprising a mixture of gamma alumina and delta alumina, said support having a surface area which ranges from 20 to 200 m²/g;
said catalyst composite being capable of providing a dehydrogenated hydrocarbon product characterized by a bromine number of at least 19.

40. The catalyst composite as claimed in claim 39, wherein said Group VIII component is selected from the group consisting of platinum and iridium and a combination thereof.

41. The catalyst composite as claimed in claim 39, wherein the particle size of the nano-sized complex is in the range of 0.4 to 2 nm, and the nano-sized complex is characterized by UV spectra as shown in FIG. 6 and said catalyst is characterized by a X-ray powder diffraction pattern as shown in FIG. 2 of the accompanying drawings.

42. The catalyst composite as claimed in claim 39, wherein the sulphur containing capping agent is selected from the group consisting of thioglycolic acid and thiomalic acid (TMA).

43. The catalyst composite as claimed in claim 39, wherein the Group IV A component is tin.

44. The catalyst composite as claimed in claim 39, wherein the proportion of delta alumina in the outer layer of the support is in the range of 15-25% with respect to the weight of the total alumina present in the core.

45. The catalyst composite as claimed in claim 39, wherein the proportions of Group VIII component, group IVA component, alkali component, group VIA component, and the halogen component in the catalyst composite are in the ranges from 0.01 to 5.0 wt %, 0.01 to 5 wt %, 0.01 to 15 wt %, 0.01 to 1 wt % and 0.05 to 0.07 wt % respectively.

46. The catalyst composite as claimed in claim 45, wherein the Group IV A component is distributed uniformly on the outer layer of the support.

47. The catalyst composite as claimed in claim 39, wherein the amount of tin expressed as Sn:Pt atomic ratio is in the range from 0.1-10 and preferably, from 0.6-4.0 in the final form of the catalyst.

48. A process for the manufacture of a catalyst composite as claimed in claim 39, comprising the following steps: preparing a spheroidal alumina support; subjecting the spheroidal alumina support to an impregnation with a first alkali metal using a first alkali metal precursor; drying and calcining the first alkali metal impregnated support; re-impregnating the first alkali impregnated support with a group VIII component; a group IVA component, a group VIA component; a halogen component, a second alkali metal and a capping agent using their respective precursors; drying and calcining the re-impregnated support and contacting the re-impregnated support with a gas stream until the halogen content of the catalyst decreases to a predetermined level to obtain a partially de-halogenated composite; reducing the partially de-halogenated composite by treating it with a stream of gas under chemically reducing conditions to obtain a catalyst composite.

49. The process as claimed in claim 48, wherein the method step of preparing a spheroidal alumina support comprises selecting an inert alpha alumina core sphere of average 1.2 mm diameter; coating the alpha alumina core with activated alumina powder and a binder till it attains an average diameter size of 1.8 mm; hydrating the coated core, heating the hydrated core at a temperature in the range of 845° C. to 855° C. in the presence of air to obtain spheroidal alumina support containing a mixture of gamma, delta and theta alumina in the outer layer.

50. The process as claimed in claim 48, wherein the re-impregnated support is treated with a gas stream until the halogen content of the catalyst is reduced to 0.07 wt %.

51. The process as claimed in claim 48, wherein the group VIII component is a mixture of platinum and iridiumor platinum, said platinum is impregnated by using chloroplatinic acid salt as its precursor by at least one technique selected from the group consisting of ‘co-gelling with the support’ and ‘chemical vapour deposition’.

52. The process as claimed in claim 48, wherein the first and second alkali metals are lithium and sodium respectively, the proportions the first and second alkali metals ranges from 0.05 to 2 wt % and 0.05 to 1.0 wt % respectively and the precursors for the first and the second alkali metals are lithium nitrate and sodium chloride respectively.

53. The process as claimed in claim 48 wherein the halogen is chlorine and it's precursor during impregnation is HCl solution, the halogen component is impregnated together with or before the addition of the group VIII component, and the Group IVA component is tin and it is impregnated on the support using aqueous solution of its precursor, stannous chloride.

54. A process for the dehydrogenation of hydrocarbons which comprises contacting said hydrocarbons with a catalytic composite as claimed in claim 39 at a temperature in the range of 400 to 800° C., pressure of from 0.1 to 10 atmospheres and a LHSV of 0.1 to 100 h⁻¹.
